(19) United States
(12) Patent Application Publication
(10) Pub. No.: US 2022/0133931 A1
(43) Pub. Date: May 5, 2022

(54) MOBILE ENVIRONMENT INTEGRATED SYSTEM FOR ELECTROSTATIC DISINFECTION, AND A METHOD

(71) Applicants: Richard Bruce Whiting, Watkinsville, GA (US); Cameron Hobbs, Watkinsville, GA (US); Michael McCabe, Pompton Plains, NJ (US); Brian Borow, Old Bridge, NJ (US)

(72) Inventors: Richard Bruce Whiting, Watkinsville, GA (US); Cameron Hobbs, Watkinsville, GA (US); Michael McCabe, Pompton Plains, NJ (US); Brian Borow, Old Bridge, NJ (US)

(21) Appl. No.: 17/514,845

(22) Filed: Oct. 29, 2021

Related U.S. Application Data
(60) Provisional application No. 63/107,257, filed on Oct. 29, 2020.

(51) Int. Cl. A61L 2/22; A61L 2/24; A61G 3/00

(52) U.S. Cl. CPC A61L 2/22; A61L 2/24; A61L 2202/25; A61L 2202/14; A61L 2202/15; A61G 3/008

(57) ABSTRACT

An electrostatic spray disinfecting system is integrated into a mobile environment to enable the mobile environment to be automatically subjected to an electrostatic spraying operation when the mobile environment is moving or stationary without the need for a human operator to participate in the spraying operation. An electrostatic sprayer of the system is mechanically coupled to a structure of the mobile environment in a preselected position relative to an internal compartment of the mobile environment. A reservoir of disinfecting material and is fluidly coupled to the electrostatic sprayer for delivering a flow of the disinfecting material to the sprayer. When an electrical switching circuit is placed in an On state, electrical power is delivered to the electrostatic sprayer to electrostatically charge the particles of the disinfecting material delivered to the electrostatic sprayer and to cause the electrostatically-charged particles to be sprayed from the electrostatic spray nozzle into the internal compartment.

### CROSS-REFERENCE TO RELATED APPLICATIONS

The present application is a nonprovisional application claiming priority to and the benefit of the filing date of U.S. provisional application Ser. No. 63/107,257, filed on Oct. 29, 2020, entitled “AMBULANCE INTEGRATED SYSTEM FOR ELECTROSTATIC DISINFECTION,” which is incorporated by reference herein.

### BACKGROUND

The need for efficient and effective disinfectant systems and methods is prevalent in the healthcare industry and travel industry, among others. The ability to quickly and effectively disinfect an ambulance is readily evident. A variety of techniques exist for disinfecting surfaces, such as, for example, spraying them with a liquid disinfecting solution and then wiping the surfaces down to remove surplus liquid from the surface as well as dirt and germs.

In addition to traditional disinfecting techniques that rely on spraying surfaces with a disinfecting liquid solution and then wiping the surfaces down, less traditional techniques that employ electrostatic spraying are also used for disinfecting surfaces. Electrostatic spray disinfecting techniques involve spraying an electrostatically-charged mist onto surfaces to be disinfected. The disinfecting solution is combined with air and atomized by an electrode inside of the sprayer to create a spray or mist of electrically-charge particles. The electrical charge on the particles in the spray or mist repel one another and actively seek out surfaces, which the particles stick to and even wrap around to coat the sides of the surfaces. This results in a generally uniform coating of disinfectant on surfaces and objects, including surfaces of awkwardly-shaped objects that are difficult to reach. Once the spray is applied, the disinfecting agent in the spray disinfects the coated surfaces.

Although electrostatic spraying systems are known in the art, they typically either require manual operation, are incapable of spraying an entire space without participation by a human operator, require a pump device to pump the chemical to be sprayed to the spray nozzle, and/or require a controller that performs a relatively complex algorithm.

With regard to electrostatic spray disinfecting systems that require participation of a human operator, such systems are not well suited for use in environments where the area to be disinfected is in motion and/or is in frequent use, such as in the patient compartment of an ambulance, for example. It can be difficult for the human operator to operate the electrostatic spray disinfecting system while the ambulance is traveling because the motion can make it difficult for the operator to maintain balance. Also, because the ambulance compartment needs to be sprayed when it is not occupied by a patient, there may not be sufficient time between transporting one patient to the hospital and loading the next patient into the ambulance compartment for the operator to properly disinfect the compartment.

A need exists for an electrostatic spray disinfecting system that is capable of being operated automatically without the need for human operator participation, that can be operated while the area to be disinfected is in motion and that is well suited for disinfecting an ambulance compartment quickly in between one patient being dropped off at a hospital and another patient being loaded into the ambulance compartment.

### SUMMARY

The present disclosure discloses an electrostatic spray disinfecting system integrated into a mobile environment to enable the mobile environment to be automatically subjected to an electrostatic spraying operation when the mobile environment is in a moving or stationary state without the need for a human operator to participate in the electrostatic spraying operation. The electrostatic spray disinfecting system comprises an electrical power source, an electrostatic sprayer, a reservoir of disinfecting material, and an electrical switching circuit. The electrostatic sprayer is mechanically coupled to a structure of the mobile environment in a preselected position relative to an internal compartment of the mobile environment. The electrostatic sprayer comprises an electrostatic spray nozzle. The preselected position is preselected to ensure that intended surfaces and objects in the internal compartment are sprayed during the electrostatic spraying operation.

The reservoir of disinfecting material is fluidly coupled to the electrostatic sprayer for delivering a flow of the disinfecting material to the electrostatic sprayer. The disinfecting material comprises particles capable of being electrostatically charged. The electrical switching circuit is electrically coupled to the electrical power source and to electrical circuitry of the electrostatic sprayer. The electrical switching circuit is configured to be activated and deactivated to place the electrical switching circuit in an On state to an Off state, respectively, and vice versa. When the electrical switching circuit is activated to place the electrical switching circuit in the On state, the electrical switching circuit allows electrical power to be delivered from the electrical power source to the electrostatic sprayer to electrostatically charge the particles of the disinfecting material delivered to the electrostatic sprayer to cause the electrostatically-charged particles to be sprayed from the electrostatic spray nozzle into the internal compartment. In the Off state, the electrical switching circuit prevents electrical power from being delivered from the electrical power source to the electrostatic sprayer.

The present disclosure discloses a method for automatically performing an electrostatic spray disinfecting operation to disinfect an internal compartment of a mobile environment while the mobile environment is in a moving or stationary state without the need for a human operator to participate in the electrostatic spraying operation. The method comprises:

equipping the mobile environment with an electrostatic spray disinfecting system comprising an electrostatic sprayer, a reservoir, an electrical power source, an activation/deactivation device, and an electrical switching circuit; and

actuating said at least a first activation/deactivation device to cause the electrical switching circuit to be placed in the On state such that the electrical switching circuit allows electrical power to be delivered from the electrical power source to the electrostatic sprayer to electrostatically charge the particles of the disinfecting material delivered to the electrostatic sprayer and to cause the electrostatically-charged particles to be sprayed from the electrostatic spray nozzle into the internal compartment.

These and other features and advantages will become apparent from the following description, drawings and claims.

### BRIEF DESCRIPTION OF THE DRAWINGS

The example or representative embodiments are best understood from the following detailed description when read with the accompanying drawing figures. It is emphasized that the various features are not necessarily drawn to scale. In fact, the dimensions may be arbitrarily increased or decreased for clarity of discussion.

FIG. 1 illustrates a block diagram of the electrostatic spray disinfecting system in accordance with a representative embodiment integrated into a mobile environment to enable an internal compartment of the mobile environment to be automatically subjected to an electrostatic spraying operation when the mobile environment is in either a moving or stationary state without the need for a human operator to participate in the electrostatic spraying operation.

FIG. 2 illustrates a top view of an exemplary application scenario for an integrated electrostatic spray disinfecting system/mobile environment in which the mobile environment is an ambulance that is integrated with the electrostatic spray disinfecting system shown in FIG. 1.

FIG. 3 illustrates a backend view of the patient compartment of the ambulance shown in FIG. 2 as a disinfectant spraying operation is being performed, where the nozzle is positioned in an upper corner of the patient compartment where the ceiling of the patient compartment meets a left side of the patient compartment and a front wall of the patient compartment that separates the patient compartment from the cab of the ambulance.

FIG. 4 is an enlarged perspective view of the nozzle shown in FIG. 3 located in the preselected position shown in FIG. 3.

FIG. 5 is a perspective view of a portion of the cab of the ambulance shown in FIG. 2 that includes a control panel on the dashboard that includes the activation/deactivation device shown in FIG. 1 to allow the driver or a passenger riding in the cab to activate/deactivate the electrical switching circuit shown in FIG. 1 to thereby automatically activate/deactivate the electrostatic sprayer shown in FIG. 1.

FIG. 6 is an enlarged perspective view of the control panel shown in FIG. 5 showing the activation/deactivation device being activated by a first responder operating the ambulance, thereby automatically activating the electrostatic sprayer shown in FIG. 1.

FIG. 7 illustrates a block diagram of the electrostatic spray disinfecting system shown in FIG. 1 in accordance with a representative embodiment that can be integrated into a mobile environment to enable an internal compartment of the mobile environment to be automatically subjected to an electrostatic spraying operation when the mobile environment is in either a moving or stationary state, without the need for a human operator to participate in the electrostatic spraying operation.

FIG. 8 is a perspective view of various components of the system shown in FIG. 7 mounted in one of the cabinets of the patient compartment of the ambulance shown in FIGS. 2 and 3.

### DETAILED DESCRIPTION

Various embodiments, aspects and features of the present disclosure encompass an electrostatic spray disinfecting system configured to be integrated into a mobile environment to enable the mobile environment to be automatically subjected to an electrostatic spraying operation when the mobile environment is in either a moving or stationary state without the need for a human operator to participate in the electrostatic spraying operation.

In the following detailed description, a few exemplary, or representative, embodiments are described to demonstrate the inventive principles and concepts. For purposes of explanation and not limitation, the representative embodiments disclose specific details in order to provide a thorough understanding of an embodiment according to the present disclosure. However, it will be understood to one having ordinary skill in the art, and having the benefit of the present disclosure, that other embodiments that depart from the specific details disclosed herein remain within the scope of the appended claims. Moreover, descriptions of well-known apparatuses and methods may be omitted to avoid obscuring the description of the representative embodiments. Such methods and apparatuses are within the scope of the present disclosure, as will be understood by those of skill in the art in view of the present disclosure.

Terminology used herein is for purposes of describing particular embodiments only, and is not intended to be limiting. The defined terms are in addition to the technical and scientific meanings of the defined terms as commonly understood and accepted in the technical field of the present teachings.

As used in the specification and appended claims, the terms “a,” “an,” and “the” include both singular and plural referents, unless the context clearly dictates otherwise. Thus, for example, “a device” includes one device and plural devices.

Relative terms, such as forwardly-facing, rearwardly facing, front, back, for example, may be used to describe the various elements' relationships to one another, as illustrated in the accompanying drawings. These relative terms are intended to encompass different orientations of the device and/or elements in addition to the orientation depicted in the drawings.

It will be understood that when an element or component is referred to as being “connected to” or “coupled to” another element, it can be directly connected or coupled, or intervening elements may be present. When an element or component is referred to as being “electrically coupled to” another element, it can be directly connected by wired connection, or intervening elements may be present, it can be connected via a wireless interface, or it can be connected via an electromagnetic interface.

Exemplary, or representative, embodiments will now be described with reference to the figures, in which like reference numerals represent like components, elements or features. It should be noted that features, elements or components in the figures are not intended to be drawn to scale, emphasis being placed instead on demonstrating inventive principles and concepts.

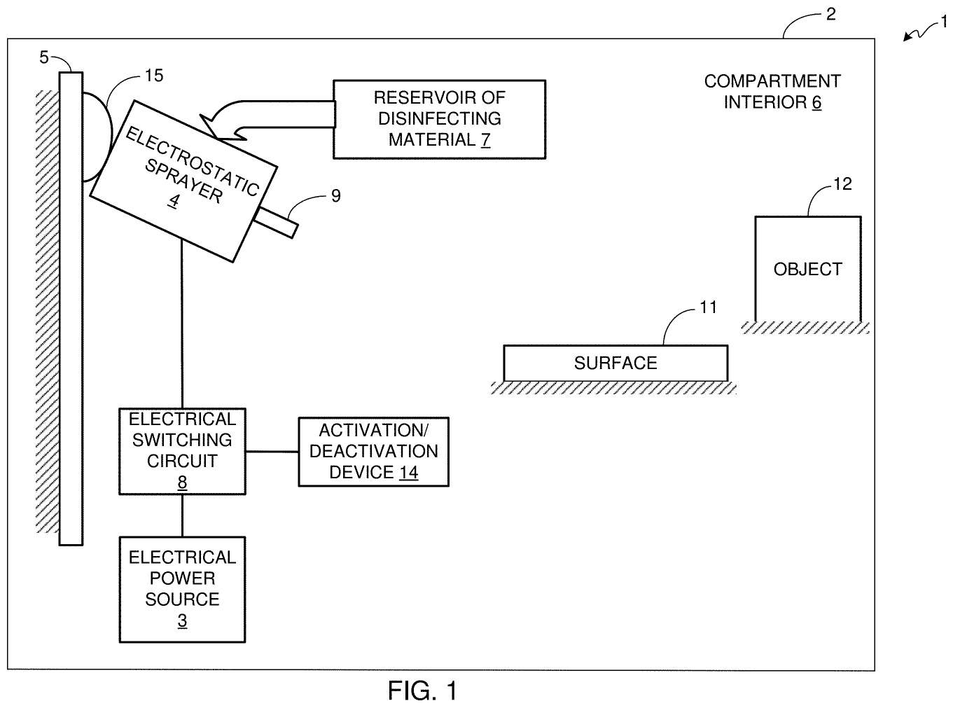

FIG. 1 is a block diagram of the electrostatic spray disinfecting system 1 in accordance with a representative embodiment integrated into a mobile environment 2 to enable an internal compartment 6 of the mobile environment 2 to be automatically subjected to an electrostatic spraying operation when the mobile environment 2 is in either a moving or stationary state without the need for a human operator to participate in the electrostatic spraying operation. The mobile environment 2 can be any mobile environment such as, but not limited to, an ambulance, a ride share vehicle, a recreational vehicle (RV), a camper, a taxicab, a police car or SUV, a military vehicle, an airplane or jet, a cruise ship, etc. The system 1 is particularly well suited for use in mobile environments that have relatively quick changeovers between persons occupying the internal compartment of the mobile environment, such as ambulances. In such environments, there can be a small window of time between the time one person is discharged or unloaded from the internal compartment and another person is loaded into the internal compartment. Embodiments of the present disclosure allow a driver or passenger of the mobile environment to cause an electrostatic spraying operation to automatically be performed even as the mobile environment is in route to pick up, for example, another rider or patient.

In accordance with this representative embodiment, the system 1 comprises an electrical power source 3, an electrostatic sprayer 4 mechanically coupled to a structure 5 of the mobile environment 2 via a coupling mechanism 15, a reservoir 7 configured to hold disinfecting material, and an electrical switching circuit 8. The electrostatic sprayer 4 is mechanically coupled to the structure 5 of the mobile environment 2 via the coupling mechanism 15 in a preselected position relative to an internal compartment 6 of the mobile environment 2. The reservoir 7 is fluidly coupled to the electrostatic sprayer 4 to enable a flow of disinfecting material to be delivered from the reservoir 7 to the electrostatic sprayer 4. The electrical switching circuit 8 is electrically coupled to the electrical power source 3 and to electrical circuitry of the electrostatic sprayer 4. The electrical switching circuit 8 can be activated and deactivated to deliver electrical power to, and to prevent electrical power from being delivered to, respectively, the electrostatic sprayer 4 from the electrical power source 3. The electrostatic sprayer 4 comprises an electrostatic spray nozzle 9 that electrically charges particles of the disinfecting material delivered from the reservoir 7 to the electrostatic sprayer 4 and sprays electrically-charged droplets into the internal compartment 6.

The preselected position at which the sprayer 4 is coupled to the structure 5 of the mobile environment 2 via the coupling mechanism 15 is preselected to ensure that surfaces 11 and objects 12 in the internal compartment 6 are effectively sprayed during the electrostatic spraying operation. The electrical switching circuit 8 is configured to be activated and deactivated to place the electrical switching circuit 8 in an On state and in an Off state, respectively, and vice versa. To accomplish the switching of the electrical switching circuit 8 from the Off state to the On state, and vice versa, the system 1 preferably includes an activation/deactivation device 14 that is electrically coupled to the electrical switching circuit 8 for switching the electrical switching circuit 8 from the Off state to the On state, and vice versa. The activation/deactivation device 14 can be, for example, a toggle switch, a pushbutton switch, a selector switch, a joystick switch, a limit switch, a proximity switch, or any other device that is suitable for manipulation by a human operator or machine. As will be described below in more detail, the activation/deactivation device 14 can be located in the cab of the mobile environment 2 (e.g., on the dashboard of a vehicle comprising the mobile environment 2) to allow the driver or a passenger in the cab to easily activate and deactivate the activation/deactivation device 14.

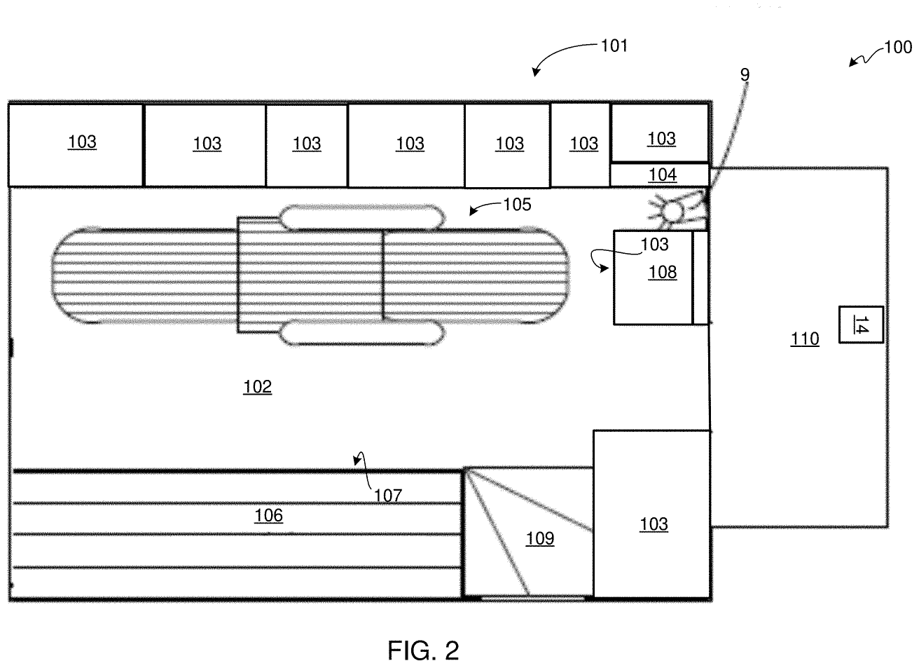

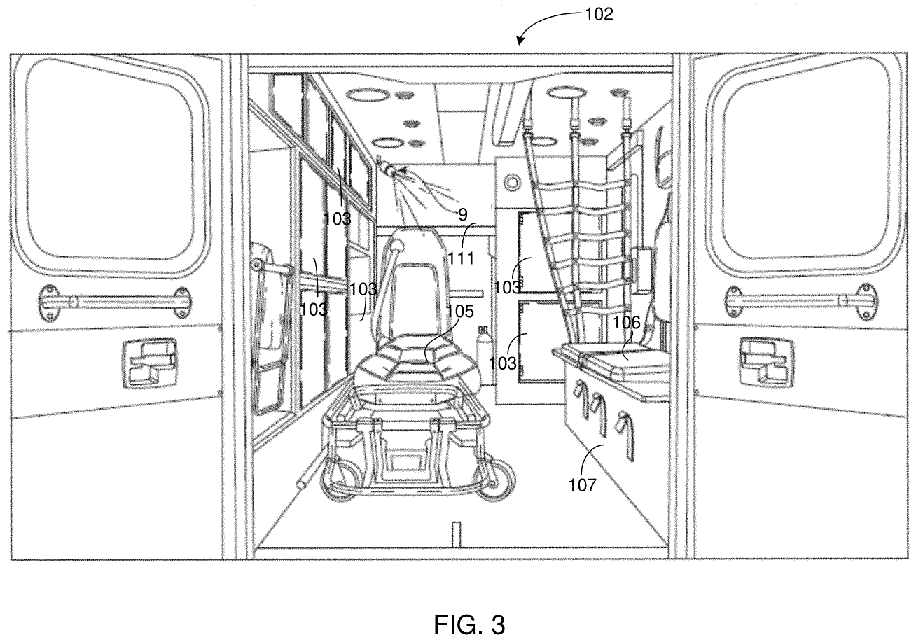

When the electrical switching circuit 8 is activated to place it in the On state, the electrical switching circuit 8 allows electrical power to be delivered from the electrical power source 3 to the electrostatic sprayer 4 to electrically charge the particles of the disinfecting material delivered from the reservoir 7 to the electrostatic sprayer 4 and to cause the electrically-charged particles to be sprayed from the electrostatic spray nozzle 9 onto surfaces 11 and objects 12 in the internal compartment 6. When the electrical switching circuit 8 is deactivated to place it in the Off state, the electrical switching circuit 8 prevents electrical power from being delivered from the electrical power source 3 to the electrostatic sprayer 4. As will be described below in more detail with reference to FIGS. 2 and 3, various components of the electrostatic sprayer 4, such as, for example, the reservoir 7, the electrical switching circuit 8, the electrical power source 3, and/or other components that may comprise the sprayer 4, are typically stored in one or more cabinets of the internal compartment of the mobile environment.

As is understood by persons of skill in the art of electrostatic spraying, an electrostatic charge may be applied by the spray nozzle 9 to an atomized flow of the disinfecting material received from the reservoir 7 and sprayed from nozzle 9 such that electrically-charged droplets of the disinfecting material are electrically attracted to surfaces 11 and objects 12 that may harbor pathogens or the like. Because a variety of electrostatic sprayers having various configurations are known in the art, the configuration of the electrostatic spray nozzle 9 and of the fluid flow mechanics of the sprayer 4 will not be described in detail herein in the interest of brevity.

During operation of the system 1, a disinfectant liquid is mixed with a pressurized air flow through an orifice in the sprayer 4 that breaks the liquid into particles. The liquid particles are entrained in the liquid flow and come into contact with an energized component that can be in the form of a cone or frustum that is in contact with an electrode inside of the sprayer 4 or within the spray nozzle 9. The energized component defines a mixing chamber. As a result, while in the mixing chamber, the particles become electrostatically charged before exiting the spray nozzle 9. In some embodiments, the sprayer 4 can include a removable cap component within which the energized component is nested. The cap component can include a pair of windows that cooperate with tabs on the body of the spray nozzle 9 in order to mechanically engage the cap to the body of the spray nozzle 9 such that the energized component is properly positioned to mate with the electrode and define the mixing chamber.

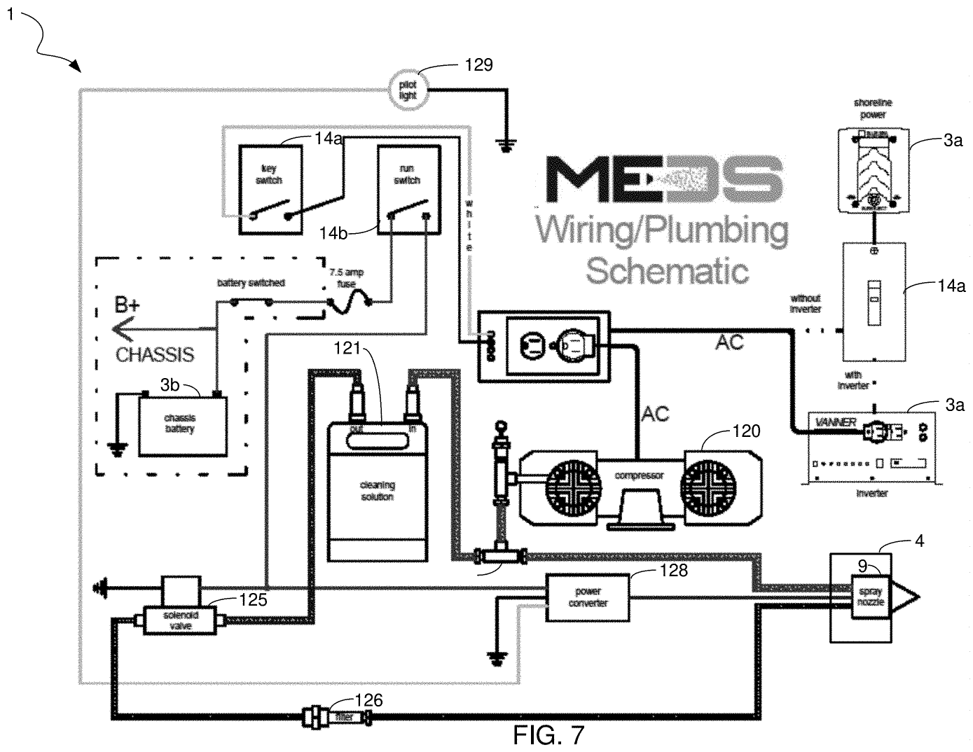

It will be understood by those of skill in the art in view of the description provided herein that the inventive principles and concepts are not limited to any particular type or configuration for the electrostatic sprayer 4 or nozzle 9. As is known in the art, electrostatic sprayers can be direct current (DC)-powered sprayers that utilize a DC battery or alternating current (AC)-powered sprayers that connect to an AC power outlet of the mobile environment 2. The latter can include an air compressor that produces pressurized air that pushes the electrically-charged particles from the nozzle 9 onto the surfaces 11 and objects 12 to reduce blowback and to increase contact between the electrically-charged particles and the surfaces 11 and objects 12, thereby improving disinfecting efficacy. The inventive principles and concepts of the present disclosure apply equal to both types of electrostatic sprayers. A preferred embodiment of the electrostatic spray disinfecting system 1 is described below with reference to FIG. 3 that uses an AC power source and a DC power source to obviate the need for a pump to pump the disinfecting material from the reservoir 7 to the sprayer 4. However, in other embodiments, a pump can be incorporated into the system 1 to obviate the need for other components, as will be discussed below in more detail with reference to FIG. 7.

In the representative embodiment depicted in FIG. 1, the electrostatic sprayer 4 can be easily and quickly activated and deactivated by an operator who manipulates the activation/deactivation device 14 to cause the electrical switching circuit 8 to deliver or prevent delivery, respectively, of electrical power to the sprayer 4 from the electrical power source 3. When electrical power is delivered to the sprayer 4, an electrical charge is placed across terminals of an electrode (not shown) disposed in the nozzle 9, which places an electrical charge on the particles of the disinfecting material as they pass through the nozzle 9. The electrically-charged droplets repel each other since the charges are of the same polarity and come into contact with the surfaces 11 and objects 12, wrapping around sides of the surfaces 11 and objects12 to coat them with disinfectant.

One advantage of embodiments of the present disclosure over prior art systems is that the sprayer 4 may be used without the need for a human operator to perform the spraying operation. The system 1, therefore, provides a way to disinfect, for example, an ambulatory patient compartment in between uses, even as the ambulance is being driven, with a high efficacy. Another advantage is that the system 1 is that it does not require a controller that performs a complex algorithm. All that is required is that a human operator or machine activate the activation/deactivation device 14 to cause electrical power to be provided to the electrostatic sprayer 4. Obviating the need for a controller reduces the cost and complexity of the system 1.

FIG. 2 illustrates a top view of an exemplary application scenario for an integrated electrostatic spray disinfecting system/mobile environment 100 in which the mobile environment is an ambulance 101 that is integrated with the electrostatic spray disinfecting system 1 shown in FIG. 1. Integration of the electrostatic spray disinfecting system 1 with the ambulance 101 enables a patient compartment 102 of the ambulance 101 to be automatically subjected to an electrostatic spraying operation when the ambulance 101 is in either a moving state or a stationary state without the need for a human operator to participate in the electrostatic spraying operation. Furthermore, the system 1 can be activated/deactivated via the activation/deactivation device 14 from the inside of the cab 110 of the ambulance 101.

In FIG. 2, the only component of the electrostatic spray disinfecting system 1 that is visible is the electrostatic nozzle 9 because the other components shown in FIG. 1 are stowed away in spaces of the patient compartment 102 that are not visible in FIG. 1, as will be discussed below in more detail.

In the representative embodiment shown in FIG. 2, the nozzle 9 is strategically mounted at a preselected location within the patient compartment 102 of the ambulance 101. The mounting location of the nozzle 9 is selected to provide the most effective and/or desirable spraying of surfaces and objects located within the patient compartment 102. The patient compartment 102 typically includes multiple cabinets 103, one or more countertops 104, one or more patient stretchers 105, a bench seat 106, storage compartments 107 underneath the bench seat 106, one or more other seats 108, and a step down 109. Of course, the inventive principles and concepts are not limited with respect to the configuration of the ambulance 101, as ambulances can have a variety of configurations.

Other components of the system 1 such as components 3, 7, 8 and 14 shown in FIG. 1, for example, can be located inside of one or more of the cabinets 103 or other spaces within the patient compartment 102 of the ambulance 101, preferably near the nozzle 9. For example, the components of the system 1 that are not visible in FIG. 2 may be mounted in or contained in a cabinet 103 underneath seat 108.

Although it is difficult to discern from the top view shown in FIG. 2, the preselected location of the nozzle 9 is near the ceiling of the patient compartment 102 in a corner behind the driver's side of the ambulance 101 to ensure that spray from the nozzle 9 effectively covers desired surfaces and objects within the compartment 102, such as at least the stretcher 105, the bench seat 106 and surrounding areas.

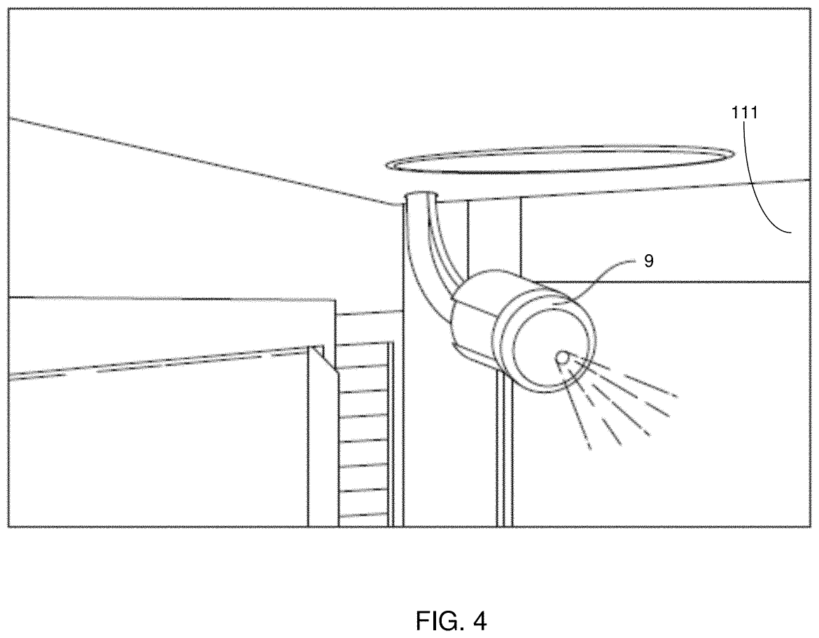

FIG. 3 illustrates a backend view of the patient compartment 102 of the ambulance 101 as a disinfectant spraying operation is being performed. In the backend view of the patient compartment 102 shown in FIG. 3, it can be seen that the nozzle 9 is positioned in an upper corner of the patient compartment 102 where the ceiling of the patient compartment 102 meets a left side of the patient compartment 102 and a front wall 111 of the patient compartment 102 that separates the patient compartment 102 from the cab 110. FIG. 4 is an enlarged perspective view of the nozzle 9 located in the preselected position shown in FIG. 3.

In this preselected position of the nozzle 9, the nozzle 9 is aimed to ensure that the electrically-charged droplets propelled or discharged from the nozzle 9 effectively coat all intended or desired surfaces and objects with disinfectant, such as the stretcher 105, bench seat 106, outer surfaces and handles of the cabinets 103, etc. As indicated above, the electrostatically-charged particles discharged from the nozzle 9 are attracted to the surfaces and objects such that the coating of disinfectant material actually wraps around surfaces to enter hard-to-reach places along the sides of the surfaces and in cracks and crevices between surfaces.

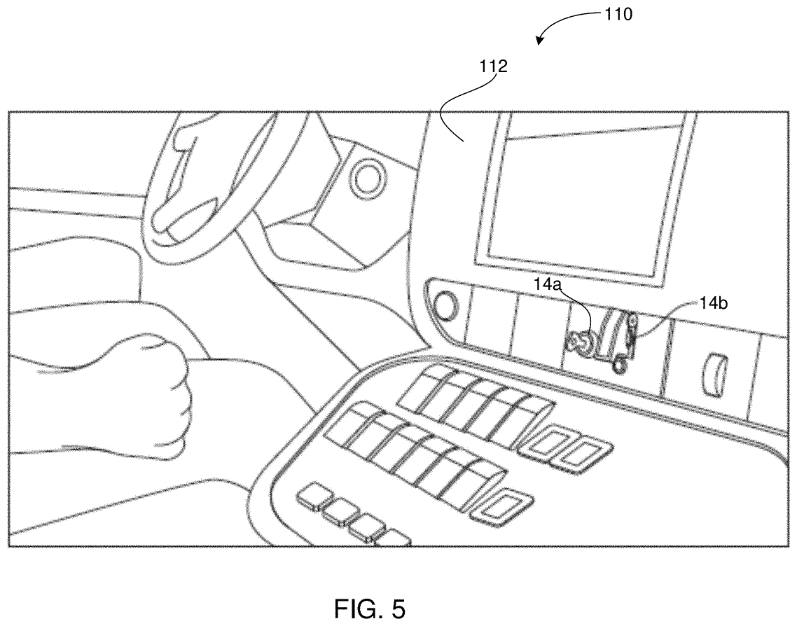

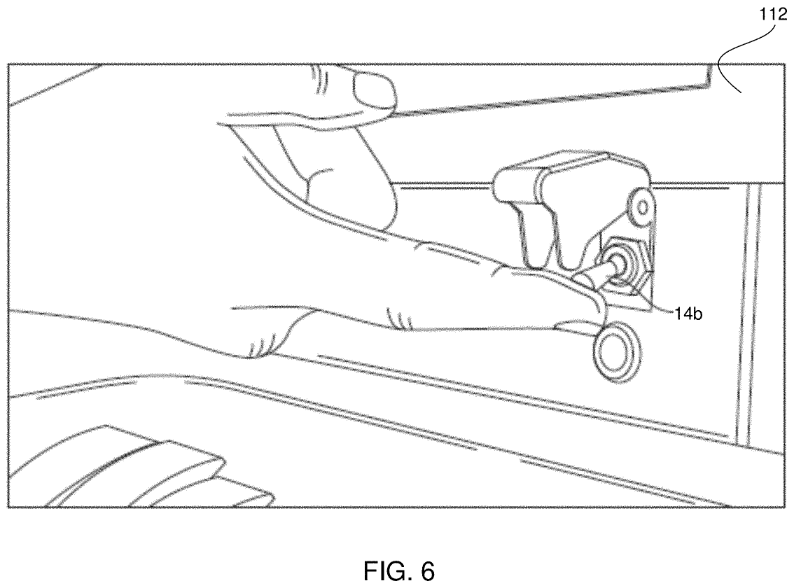

FIG. 5 is a schematic view of a portion of the cab 110 that includes a control panel 112 on the dashboard that includes the activation/deactivation device 14 shown in FIG. 1 to allow the driver or a passenger riding in the cab 110 to activate/deactivate the electrical switching circuit 8 shown in FIG. 1 to thereby automatically activate/deactivate the electrostatic sprayer 4 shown in FIG. 1. FIG. 6 is an enlarged schematic view of the control panel 112 shown in FIG. 4 showing the activation/deactivation device 14 being activated by a first responder operating the ambulance 101, thereby automatically activating the electrostatic sprayer 4.

In accordance with this representative embodiment, the activation/deactivation device 14 comprises a first activation/deactivation device 14a and a second activation/deactivation device 14b for causing AC power and DC power, respectively, to be supplied to the electrostatic sprayer 14. As will be described below in detail with reference to FIG. 7, when the system 1 is integrated with an ambulance, the electrical power source 3 comprises an AC power supply and a DC power supply. In such an embodiment, activation of the first activation/deactivation device 14a places an AC power switching configuration of the electrical switching circuit 8 in an On state that allows AC power to be delivered to one or more components of the system 1. While the AC power switching configuration is in the On state, activation of the second activation/deactivation device 14a places a DC power switching configuration of the electrical switching circuit 8 in an On state that allows DC power to be delivered to one or more components of the system 1, including the sprayer 4, to cause an electrostatic spraying operation to be performed.

In accordance with this embodiment, a key switch 14a is turned in the clockwise direction to supply AC power to the system 1 and a toggle switch 14b is flipped to the up position to supply DC power to the system and to activate the sprayer 4. Thus, the entire patient compartment 102 can be automatically disinfected by a simple “turn of the key” and “flip of the switch” from inside of the cab 110, even as the ambulance 101 is driving down the road. As indicated above, a variety of activation/deactivation mechanism can be used for this purpose. The two devices shown are merely examples of suitable mechanisms for this purpose.

FIG. 7 illustrates a block diagram of the electrostatic spray disinfecting system 1 shown in FIG. 1 in accordance with a representative embodiment that can be integrated into a mobile environment to enable an internal compartment of the mobile environment to be automatically subjected to an electrostatic spraying operation when the mobile environment is in either a moving or stationary state, without the need for a human operator to participate in the electrostatic spraying operation. In the representative embodiment shown in FIG. 7, the mobile environment is not shown so as not to obscure details of the system 1.

In accordance with this representative embodiment, the electrical power source 3 depicted in FIG. 1 comprises an AC power supply 3a and a DC power supply 3b. The AC power supply 3a can be an on-board AC power supply, such as, for example, the power inverter shown in FIG. 7, which has its own DC power supply from which the inverter generates an 110 V AC signal, or the AC power supply 3a can be external to the system 1, such as, for example, an AC power outlet provided in the ambulance parking area of a hospital. Such an external AC power outlet is typically referred to as shoreline power. In the case where shoreline power is used, a toggle switch 14a that is external to the system 1 can be used to turn the AC power supply on and off. When the inverter 3a is used, a toggle switch 14a of the system 1 is used to turn the Ac power on and off.

In accordance with this representative embodiment, the system 1 comprises an air compressor 120, a pressurized reservoir 121 that acts as the reservoir 7 shown in FIG. 1, a control valve 125 (e.g., a solenoid switch), a fluid control system 126 and the electrostatic sprayer 4 with spray nozzle 9. Preferably the system 1 also includes a DC-to-DC converter 128 that converts a 12 V DC signal from the DC power supply 3b into a high-voltage (e.g., 1200 to 1500 V) signal for electrostatically charging the particles of disinfectant material in the nozzle 9.

As indicated above, electrostatic disinfecting systems can be DC power or AC powered, so in some embodiments, the system 1 can include the DC power supply 3b, but not the AC power supply 3a, while in other embodiments, the system 1 can include the AC power supply 3a, but not the DC power supply 3b. Furthermore, in some embodiments, the system 1 can include an AC-to-DC converter and/or a DC-to-AC converter, such as inverter 3a, to convert an AC supply signal into a DC signal, respectively.

In the preferred, or exemplary, embodiment shown in FIG. 7, the system 1 is configured for integration into an ambulance and preferably includes the AC power supply 3a comprising the inverter and the DC power supply 3b for reasons described below in more detail. The AC power supply 3a can be, for example, a 110 V or 120 V wall socket. Ambulances commonly include such wall sockets in the patient compartment. Ambulances also commonly include both AC and DC power supplies. The key switch 14a shown in FIG. 5 can be used to “start” the system 1 and then the toggle switch 14b can be used to start the disinfecting spray operation to automatically distribute the electrostatically-charged particles or droplets within the ambulatory patient compartment. As indicated above, the driver or a passenger in the cab 110 can actuate the system 1 while the ambulance is being driven and the patient compartment is not in use.

Starting the system 1 by turning the key switch 14a in the clockwise direction to the start position causes the aforementioned AC power switching configuration of the electrical switching circuit 8 to deliver AC power from the AC power supply to the air compressor 120. The air compressor 120 supplies compressed air to the electrostatic spray nozzle 9 via one or more air hoses or lines. A splitter 122 splits the flow of compressed air passing out of an air output port of the air compressor 120 into first and second air flows and air hoses carry the first and second air flows to an air input port of the reservoir 121 and to an air input port of the sprayer 4. The control valve 125 preferably is a “normally closed” arrangement. The control valve 125 can work to prevent or mitigate “leak by” due to residual system pressure when the system 1 is turned off.

After the system 1 has been turned on to provide AC power to the air compressor 120, a disinfecting spray operation can be commenced by turning the DC power switch 14b to the On position, which in this embodiment is performed by flipping the toggle switch shown in FIG. 6 to the up position. In this position, the DC power switching configuration of the electrical switching circuit 8 shown in FIG. 1 causes DC power to be delivered to the sprayer 4 via the DC-DC converter 128 and also activates the control valve 125 to allow pressurized disinfecting material to flow into the fluid control system 126. The fluid control system 126 performs filtering, air flow restriction and other tasks to control the parameters of the flow of disinfecting material into the sprayer 4, as is known in the art. The disinfecting material in the pressurized reservoir 121 is supplied via one or more fluid supply lines (not shown) from a fluid output port of the reservoir 121 to a fluid input port of the fluid control system 126. Likewise, the flow of disinfecting material is supplied via one or more fluid supply lines (not shown) from a fluid output port of the fluid control system 126 to a fluid input port of the electrostatic sprayer 4.

During operation of the system 1, the disinfectant liquid received in the sprayer 4 is mixed with the pressurized air flow supplied via air lines from the air compressor 120 to an orifice in the sprayer 4. The pressurized air flow breaks the disinfectant liquid into liquid particles. The liquid particles are entrained in the liquid flow and come into contact with an energized component (e.g., a cone or frustum) that is in contact with an electrode (not shown) inside of the sprayer 4 or inside of the spray nozzle 9. The energized component defines a mixing chamber in which the particles become electrostatically charged before being discharged from the spray nozzle 9. As indicated above, in some embodiments the sprayer 4 can include a removable cap component within which the energized component is nested. The cap component can include a pair of windows that cooperate with tabs on the body of the spray nozzle 9 in order to mechanically engage the cap to the body of the spray nozzle 9 to ensure that the energized component is properly positioned to mate with the electrode and define the mixing chamber. An indicator light 129 that turns on when the DC power supply 3b is in the On state can be used to inform personnel that a disinfecting spray operation is in process.

A variety of variations can be made to the system 1 shown in FIG. 7 and components can be added to or removed from the system 1 depending on the environment or application. For example, in some embodiments, a piston or other mechanism of actuation may be used to translate the spray nozzle 9 up and down and/or side to side as it sprays in order to ensure optimal coverage. Such techniques can be used to apply the electrostatically-charged liquid around and throughout the entire space of the patient compartment without the need for manual application. As another example, an alternative to pressurizing the reservoir 121 is to use a pump to move disinfectant material from a non-pressurized reservoir to the sprayer 4. Persons of skill in the art will understand the manner in which these and other modifications or variations can be made to the system 1 shown in FIG. 7.

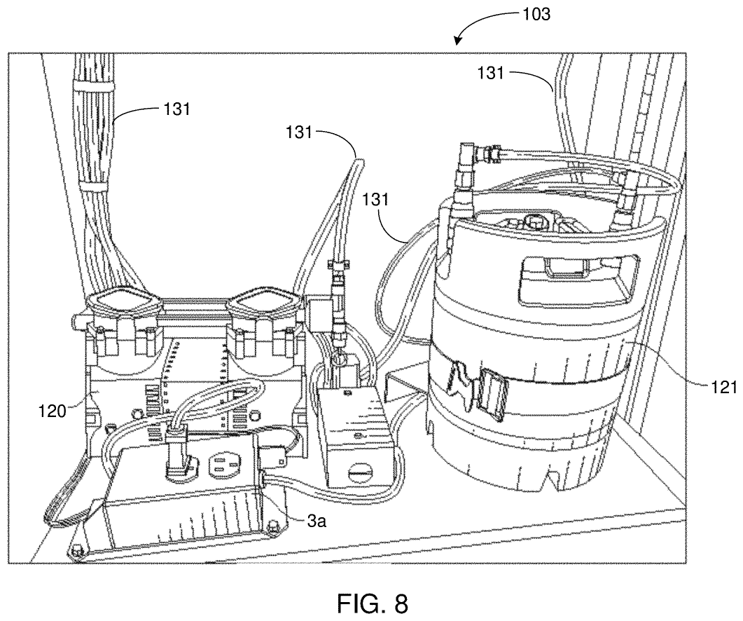

FIG. 8 is a perspective view of various components of the system 1 shown in FIG. 7 mounted in one of the cabinets 103 of the patient compartment 102 shown in FIGS. 2 and 3. Visible in FIG. 8 are, for example, the AC power supply 3a, the air compressor 120, the pressurized reservoir 121, and various lines 131, such as air supply lines, fluid supply lines and electrical cabling. In this embodiment, the cabinet 103 in which these components are mounted is located below and in close proximity to the sprayer 4 and the nozzle 9. Thus, most or all of the components of the system 1 other than the sprayer 4 and its nozzle 9 can be stowed away neatly inside of the internal compartment, and the system 1 can be easily and conveniently activated from within the cab of the mobile environment while the mobile environment is unoccupied and is either moving or stationary, without the need for human intervention to perform the spraying operation.

It should be noted that inventive principles and concepts of the present disclosure have been described with reference to a few representative, illustrative and/or preferred embodiments for the purpose of demonstrating the principles and concepts. Persons of skill in the art will understand, in view of the present disclosure, the manner in which the inventive principles and concepts can be applied to other embodiments not explicitly described herein. For example, while a particular configuration of the system 1 is described herein and shown in the figures, modifications to the configuration, including those mentioned above, can also be used, as will be understood by those skilled in the art in view of the description provided herein. Many modifications in addition to those specifically mentioned or discussed herein may be made while still achieving the goals of the inventive principles and concepts, and thus all such modifications are within the scope of the invention.

What is claimed is:

1. An electrostatic spray disinfecting system integrated into a mobile environment to enable the mobile environment to be automatically subjected to an electrostatic spraying operation when the mobile environment is in a moving or stationary state without the need for a human operator to participate in the electrostatic spraying operation, the electrostatic spray disinfecting system comprising:
an electrical power source;
an electrostatic sprayer mechanically coupled to a structure of the mobile environment in a preselected position relative to an internal compartment of the mobile environment, the electrostatic sprayer comprising an electrostatic spray nozzle, the preselected position being preselected to ensure that intended surfaces and objects in the internal compartment are sprayed during the electrostatic spraying operation;
a reservoir of disinfecting material fluidly coupled to the electrostatic sprayer for delivering a flow of the disinfecting material to the electrostatic sprayer, the disinfecting material comprising particles capable of being electrostatically charged; and
an electrical switching circuit electrically coupled to the electrical power source and to electrical circuitry of the electrostatic sprayer, the electrical switching circuit being configured to be activated and deactivated to place the electrical switching circuit in an On state to an Off state, respectively, and vice versa, wherein when the electrical switching circuit is activated to place the electrical switching circuit in the On state, the electrical switching circuit allows electrical power to be delivered from the electrical power source to the electrostatic sprayer to electrostatically charge the particles of the disinfecting material delivered to the electrostatic sprayer to cause the electrostatically-charged particles to be sprayed from the electrostatic spray nozzle into the internal compartment, and wherein in the Off state, the electrical switching circuit prevents electrical power from being delivered from the electrical power source to the electrostatic sprayer.

2. The system of claim 1, further comprising:
at least a first activation/deactivation device adapted to be actuated by a person or machine, wherein when said at least a first activation/deactivation device is actuated, the electrical switching circuit is placed in the On state such that electrical power is delivered from the electrical power source to the electrostatic sprayer to electrostatically charge the particles of the disinfecting material delivered to the electrostatic sprayer and cause the electrostatically-charged particles to be sprayed from the electrostatic spray nozzle into the internal compartment.

3. The system of claim 2, wherein the mobile environment comprises an ambulance and the internal compartment is a patient compartment of the ambulance, and wherein said at least a first activation/deactivation device is located on a control panel that is disposed in a cab of the ambulance to allow a driver of the ambulance or passenger riding in the cab to easily access said at least a first activation/deactivation device to actuate said at least a first activation/deactivation device when the ambulance is either moving or stationary.

4. The system of claim 2, wherein the mobile environment comprises a motor vehicle and the internal compartment is a passenger ride area of the motor vehicle, and wherein said at least a first activation/deactivation device is located on a control panel that is disposed in a driver area of the motor vehicle to allow a driver of the motor vehicle to easily access said at least a first activation/deactivation device to actuate said at least a first activation/deactivation device when the motor vehicle is either moving or stationary.

5. The system of claim 1, wherein the electrical power source comprises an alternating current (AC) power source.

6. The system of claim 1, wherein the electrical power source comprises a direct current (DC) power source.

7. The system of claim 1, wherein the electrical power source comprises an alternating current (AC) power source and a direct current (DC) power source.

8. The system of claim 7, wherein the electrical switching circuit comprises an AC power switching configuration and a DC power switching configuration, the system further comprising:
a first activation/deactivation device and a second activation/deactivation device, the first activation/deactivation device controlling the AC power switching configuration to control delivery of AC power from the AC power source to the system, the second activation/deactivation device controlling the DC power switching configuration to control delivery of DC power from the DC power source to the system.

9. The system of claim 8, wherein the system further comprises:
an air compressor electrically coupled to the AC power source via the AC power switching configuration of the electrical switching circuit, wherein activation of the first activation/deactivation device places the AC power switching configuration in an On state to allow AC power to be delivered from the AC power source to the air compressor, thereby causing a pressurized flow of air to be delivered from an air output port of the air compressor to an air input port of the electrostatic sprayer.

10. The system of claim 9, wherein the reservoir is a pressurized reservoir, and wherein the system further comprises:
a fluid control system having a fluid output port that is fluidly coupled with a fluid input port of the electrostatic sprayer;
a control valve electrically coupled with the DC power supply, fluidly coupled with a fluid output port of the pressurized reservoir, and fluidly coupled with a fluid input port of the fluid control system;
wherein activation of the second activation/deactivation device while the AC power switching configuration is in the On state places the DC power switching configuration of the electrical switching circuit in an On state to allow DC power to be delivered from the DC power supply to the electrostatic sprayer and to the control valve, thereby energizing a mixing chamber of the electrostatic sprayer and causing the control valve to permit a flow of disinfecting material to flow from the fluid output port of the pressurized reservoir into the fluid input port of the fluid control system, the fluid control system controlling parameters of the flow of disinfecting material output from the fluid output port of the fluid control system and input to the fluid input port of the electrostatic sprayer, and wherein the flow of the disinfecting material input to the fluid input port of the electrostatic sprayer mixes with the pressurized flow of air delivered from the air compressor to the electrostatic sprayer in the energized mixing chamber to electrostatically charge the particles of the disinfecting material before the electrostatically-charged particles are discharged from the electrostatic spray nozzle.

11. The system of claim 10, further comprising a DC-to-DC converter, wherein the DC power supply is electrically coupled to the electrostatic sprayer via the DC power electrical switching configuration of the electrical switching circuit, and wherein when the DC power switching configuration of the electrical switching circuit in the On state, a DC power signal received in the DC-to-DC converter from the DC power supply is converted into a higher voltage DC power signal that is delivered to the electrostatic sprayer and used to energize the mixing chamber.

12. The system of claim 9, wherein the reservoir is a non-pressurized reservoir, and wherein the system further comprises:
a pump electrically coupled via the electrical switching circuit to at least one of the AC power supply and the DC power supply, the pump being fluidly coupled with a fluid output port of the reservoir and with a fluid input port of the electrostatic sprayer, wherein activation of the electrical switching circuit causes the pump to pump the flow of disinfecting material from the fluid output port of the reservoir to the fluid input port of the electrostatic sprayer; and
wherein activation of the second activation/deactivation device while the AC power switching configuration is in the On state places the DC power switching configuration of the electrical switching circuit in an On state to allow DC power to be delivered from the DC power supply to the electrostatic sprayer, thereby energizing a mixing chamber of the electrostatic sprayer that mixes the flow of disinfecting material pumped from the fluid output port of the reservoir to the fluid input port of the electrostatic sprayer with the pressurized flow of air delivered from the air compressor to the electrostatic sprayer to electrostatically charge the particles of the disinfecting material before the electrostatically-charged particles are discharged from the electrostatic spray nozzle.

13. The system of claim 12, further comprising a DC-to-DC converter, wherein the DC power supply is electrically coupled to the electrostatic sprayer via the DC power electrical switching configuration of the electrical switching circuit, and wherein when the DC power switching configuration of the electrical switching circuit is in the On state, a DC power signal received in the DC-to-DC converter from the DC power supply is converted into a higher voltage DC power signal that is delivered to the electrostatic sprayer and used to energize the mixing chamber.

14. A method for automatically performing an electrostatic spray disinfecting operation to disinfect an internal compartment of a mobile environment while the mobile environment is in a moving or stationary state without the need for a human operator to participate in the electrostatic spraying operation, the method comprising:
equipping the mobile environment with an electrostatic spray disinfecting system, the electrostatic spray disinfecting system comprising an electrostatic sprayer, a reservoir, an electrical power source, an activation/deactivation device, and an electrical switching circuit, the electrostatic sprayer being mechanically coupled to a structure of the mobile environment in a preselected position relative to the internal compartment of the mobile environment, the electrostatic sprayer comprising an electrostatic spray nozzle, the preselected position being preselected to ensure that intended surfaces and objects in the internal compartment are sprayed during the electrostatic spraying operation, the reservoir holding disinfecting material and being fluidly coupled to the electrostatic sprayer for delivering a flow of the disinfecting material to the electrostatic sprayer, the disinfecting material comprising particles capable of being electrostatically charged, the electrical switching circuit being electrically coupled to the electrical power source and to electrical circuitry of the electrostatic sprayer, the electrical switching circuit being configured to be activated and deactivated through actuation and deactuation, respectively, of said at least a first activation/deactivation device to place the electrical switching circuit in an On state to an Off state, respectively, and vice versa; and
actuating said at least a first activation/deactivation device to cause the electrical switching circuit to be placed in the On state such that the electrical switching circuit allows electrical power to be delivered from the electrical power source to the electrostatic sprayer to electrostatically charge the particles of the disinfecting material delivered to the electrostatic sprayer and to cause the electrostatically-charged particles to be sprayed from the electrostatic spray nozzle into the internal compartment.

15. The method of claim 14, wherein the electrical power source comprises an alternating current (AC) power source and a direct current (DC) power source, and wherein the electrical switching circuit comprises an AC power switching configuration and a DC power switching configuration, said at least a first activation/deactivation device comprising first and second activation/deactivation devices, the first activation/deactivation device controlling the AC power switching configuration to control delivery of AC power from the AC power source to the system, the second activation/deactivation device controlling the DC power switching configuration to control delivery of DC power from the DC power source to the system, the electrostatic spray disinfecting system further comprising an air compressor electrically coupled to the AC power source via the AC power switching configuration of the electrical switching circuit, the method further comprising:
actuating the first activation/deactivation device to place the AC power switching configuration in an On state to allow AC power to be delivered from the AC power source to the air compressor, thereby causing a pressurized flow of air to be delivered from an air output port of the air compressor to an air input port of the electrostatic sprayer.

16. The method of claim 15, wherein the reservoir is a pressurized reservoir, and wherein the electrostatic spray disinfecting system further comprises a fluid control system and a control valve, the fluid control system having a fluid output port that is fluidly coupled with a fluid input port of the electrostatic sprayer, the control valve being electrically coupled with the DC power supply, fluidly coupled with a fluid output port of the pressurized reservoir, and fluidly coupled with a fluid input port of the fluid control system, the method further comprising:
while the AC power switching configuration is in the On state, actuating the second activation/deactivation device to place the DC power switching configuration of the electrical switching circuit in an On state to allow DC power to be delivered from the DC power supply to the electrostatic sprayer and to the control valve, thereby energizing a mixing chamber of the electrostatic sprayer and causing the control valve to permit a flow of disinfecting material to flow from the fluid output port of the pressurized reservoir into the fluid input port of the fluid control system, the fluid control system controlling parameters of the flow of disinfecting material output from the fluid output port of the fluid control system and input to the fluid input port of the electrostatic sprayer, and wherein the flow of the disinfecting material input to the fluid input port of the electrostatic sprayer mixes with the pressurized flow of air delivered from the air compressor to the electrostatic sprayer in the energized mixing chamber to electrostatically charge the particles of the disinfecting material before the electrostatically-charged particles are discharged from the electrostatic spray nozzle.

17. The method of claim 16, wherein the electrostatic spray disinfecting system further comprises a DC-to-DC converter, wherein the DC power supply is electrically coupled to the electrostatic sprayer via the DC power electrical switching configuration of the electrical switching circuit, and wherein when the DC power switching configuration of the electrical switching circuit is in the On state, a DC power signal received in the DC-to-DC converter from the DC power supply is converted into a higher voltage DC power signal that is delivered to the electrostatic sprayer and used to energize the mixing chamber.

18. The method of claim 15, wherein the reservoir is a non-pressurized reservoir, and wherein the electrostatic spray disinfecting system further comprises a pump that is electrically coupled via the electrical switching circuit to at least one of the AC power supply and the DC power supply, the pump being fluidly coupled with a fluid output port of the reservoir and with a fluid input port of the electrostatic sprayer, the method further comprising:
while the AC power switching configuration is in the On state and AC power is being delivered from the AC power source to the air compressor to cause a pressurized flow of air to be delivered from an air output port of the air compressor to an air input port of the electrostatic sprayer, actuating the pump to cause the pump to pump the flow of disinfecting material from the fluid output port of the reservoir to the fluid input port of the electrostatic sprayer; and
while the pressurized flow of air and the flow of disinfecting material are being delivered and pumped, respectively, to the air input port and the fluid input port of the electrostatic sprayer, respectively, actuating the second activation/deactivation device to place the DC power switching configuration of the electrical switching circuit in the On state to allow DC power to be delivered from the DC power supply to the electrostatic sprayer, thereby energizing a mixing chamber of the electrostatic sprayer that mixes the flow of disinfecting material pumped from the fluid output port of the reservoir to the fluid input port of the electrostatic sprayer with the pressurized flow of air delivered from the air compressor to the electrostatic sprayer to electrostatically charge the particles of the disinfecting material before the electrostatically-charged particles are discharged from the electrostatic spray nozzle.

19. The method of claim 14, wherein the mobile environment comprises an ambulance and the internal compartment is a patient compartment of the ambulance, and wherein said at least a first activation/deactivation device is located on a control panel that is disposed in a cab of the ambulance to allow a driver of the ambulance or passenger riding in the cab to easily access said at least a first activation/deactivation device to actuate said at least a first activation/deactivation device when the ambulance is either moving or stationary.

20. The method of claim 14, wherein the mobile environment comprises a motor vehicle and the internal compartment is a passenger ride area of the motor vehicle, and wherein said at least a first activation/deactivation device is located on a control panel that is disposed in a driver area of the motor vehicle to allow a driver of the motor vehicle to easily access said at least a first activation/deactivation device to actuate said at least a first activation/deactivation device when the motor vehicle is either moving or stationary.
